(19) United States
(12) Patent Application Publication
(10) Pub. No.: US 2003/0087753 A1
(43) Pub. Date: May 8, 2003

(54) System for microbial control of a fluid

(76) Inventors: Birger Hjertman, Vallingby (SE); Hakan Eriksson, Lund (SE); Sten Andersson, Angelholm (SE)

Correspondence Address: DINSMORE & SHOHL, LLP, 1900 CHEMED CENTER, 255 EAST FIFTH STREET, CINCINNATI, OH, 45202, US

(21) Appl. No.: 10/184,006

(22) Filed: Jun. 26, 2002

Related U.S. Application Data
(60) Provisional application No. 60/304,690, filed on Jul. 11, 2001.

(30) Foreign Application Priority Data
Jun. 26, 2001 (SE) 0102265-6

(51) Int. Cl. B01J 29/06

(52) U.S. Cl. 502/64

(57) ABSTRACT

An arrangement for controlling microbial content or growth in a fluid, comprising a) a zeolite with micro-pores charged with an agent having affinity to the zeolite micro-pores and having antiseptic properties and b) the fluid, the zeolite and the fluid being enclosed by, at least partially in contact, or being arranged for being brought at least partially in contact. The fluid contains molecules being larger than the micro-pores of the zeolite and having less affinity, as defined, to the zeolite than the agent. The fluid may contain a therapeutically active compound or composition in a therapeutically effective amount and concentration and that the total amount of agent in the zeolite is larger than an amount corresponding to an antiseptic level for the fluid. Also disclosed are methods, uses and zeolites for control of microbial content or growth in a fluid.

### TECHNICAL FIELD

The present invention relates to an arrangement for controlling microbial content or growth in a fluid. The arrangement comprises a) a zeolite with micro-pores charged with an agent having affinity to the zeolite micro-pores and having properties and b) the fluid, the zeolite and the fluid being at least partially in contact, or being arranged for being brought at least partially in contact. The invention also relates to a suitable zeolite for the arrangement and methods for use thereof.

### BACKGROUND

Use of antiseptic agents for control of microbial content or growth is common for various purposes such as cleaning and sterilizing the fluid and as preservative additives to the fluid. A general problem is that the agents tend to be toxic or hazardous not only to the microbes but to other life forms as well, including humans and animals, and it is desirable to limit the amounts of agents to a minimum. On the other hand the antiseptics frequently have to be used in excess, corresponding to the worst microbial exposure foreseen for the fluid in its contemplated use. The problems are exacerbated when the fluid is intended for body exposure, e.g. as breathing air or preparations for body treatment. Most so for injection preparations where the antiseptic type and amount are severely restricted but still have inevitable side effects which have to be balanced against the therapeutic value of the preparation treatment.

Efforts have been made to control the antiseptic amount and exposure pattern better. One suggestion is to absorb the antiseptic in a carrier to control its release rate. The EP 301717 patent specification proposes coating medical tubes with a zeolite charged with metal ions such as Ag, Cu or Zn as anti-bacterial agents for the extended prevention of infection in body tissues surrounding the inserted tube. The degree of control obtained, however, is limited. The release is entirely dictated by diffusion and cannot be varied or positively controlled. The effect extends only to a thin layer and also the zeolite has to be applied in a thin layer for efficient utilization. The ion exchange system proposed limits the absorbed agents to certain metal ions. Similar problems are encountered when using a zeolite as absorbent for a medical to be administered orally, as exemplified by EP 240169, where peak concentrations can be avoided but no control is available for obtaining low and uniform concentrations.

The WO 97/15391 reference suggests use of zeolite to absorb preservatives from pharmaceutical preparations in connection with ejection to reduce the amount of preservatives delivered to the body. The zeolite can be arranged in the front of a syringe type device to be passed by the preparation in connection with ejection. However, the position of the zeolite between the preparation and an exit opening for the preparation means that necessarily there is a low content of preservative at the most probable infection path, i.e. through the exit opening, and if this part is infected enough preservative is not available for control of its growth. The problem will be more pronounced in multi-dose arrangements since the liquid in dead spaces in front of the zeolite absorbent has passed the zeolite and is subject to uncontrolled microbial growth.

A similar problem is presented in WO 87/05592 with respect to wastewater treatment. Here the recipient to be protected is the biological bed used for breakdown of the waste content in the water. A zeolite bed is inserted in the influx line before the bed and is able to absorb occasional bursts of toxic components in the incoming water. The concentration of toxic substances is thus kept low and not higher than the biological bed is able to degrade. Since this system simply absorbs random toxic components in the incoming wastewater there is no use at all, and still less an efficient use, of the toxic component for antiseptic purposes and no control means are provided for such purposes.

Accordingly there remains a need for improved methods and means relating to control of microbial content or growth in a fluid.

### SUMMARY OF INVENTION

A main object of the present invention is to provide a system for controlling microbial growth in fluids avoiding the disadvantages and shortcomings of hitherto used technologies. A more specific object is to provide such a system based on controlled and efficient use of an antiseptic agent. Another object is provide a system allowing reduced amounts of agent with maintained antiseptic action or improved antiseptic action with maintained agent amounts. Still another object is to offer a system exposing the fluid to agent amounts adapted to its current microbial charge and to reduce the need for surplus agent amounts for worst case purposes. A further object is to offer a system providing an antiseptic barrier between a confined fluid and the environment, independent of the fluid content of agent. Yet another object is to provide a system giving treated fluid of sufficiently low agent content to permit its exposure to human and animals, including injection into the body, and compatible with fluids being pharmaceutical preparations. A further object is to offer a system applicable to both small and large volumes of fluid in static as well as intermittent or continuous movement or dosing. Yet another object is to offer a system allowing use of a broad range of antiseptic agents. A further object is to offer a system usable for both gas and liquid fluids. Still another object is to provide a system allowing devices and arrangements of flexible design.

These objects are reached with the characteristics set forth in the appended patent claims.

In the system of the invention a zeolite is used for absorption of the antiseptic agent. Compared with other absorbents zeolites are generally highly inert and structurally rigid, can be shaped into structures of very flexible form and varying bulk porosity, and are selective and efficient absorbents due to the high pore contents and uniform pore sizes, yet with a possibility to vary the pore size for adaptation to specific target molecules. Additional advantages of importance in the present context are obtained with hydrophobic zeolites, i.e. zeolites with high silicon and low aluminum content in the crystal lattice backbone. These zeolites are still more inert and stable, of particular importance where the fluid is to be exposed to human or animals. They have no or low tendency to release particles and aluminum ions, of importance for example when the fluid is a pharmaceutical preparation. They sustain high temperatures and prolonged exposure to water without degradation, of importance for example to allow sterilization and long storage periods of pre-loaded devices or repeated operation or regeneration in continuous cleaning arrangements. Finally the hydrophobic zeolites broaden the range of possible antiseptics by being compatible also with hydrophobic and non-ionic compounds, which is of importance these classes of compounds cover many antiseptics permitted for human exposure and which operates particularly well with the principles of the present invention. The invention utilizes a zeolite pre-charged with an antiseptic agent to such a level that fluid in contact with the pre-charged zeolite will attain an antiseptic level of the agent by controlled release from the zeolite. Accordingly the invention utilizes a hitherto not exploited property of the zeolites, namely their ability to absorb such quantities of an agent that a fluid in contact with the zeolite increases its content of agent to an antiseptic level. This in contrast e.g. with zeolite applications for filtering where the fluid will have its agent content reduced from antiseptic level to lower than antiseptic levels under concurrent increase of agent to moderate levels in the zeolite filter. This new way of using the zeolite meets several objects of the invention. The levels of antiseptic agent in the fluid can be kept low and need not be raised to excess levels for worst case situations since the charged zeolite acts as a buffer, providing additional release of agent when needed, e.g. when new fluid volumes are contacted or agent is consumed by microbes or otherwise. Without being bound by theory it is hypothesized that the fluid/zeolite system tries to establish an equilibrium with certain levels of agent in the fluid and in the zeolite respectively and that any disturbance leads to a drive towards a new equilibrium by release or uptake of agent by the zeolite. Accordingly a consumption controlled low and always appropriate level of agent in the fluid can be maintained in spite of the larger amounts absorbed to the zeolite, also allowing repeated equilibrium to be established with at least quasi-static fluid concentrations of agent. The fluid and zeolite can be contacted statically with a zeolite body or powder, by passing the fluid past a zeolite surface or coating or through a bed or column of the zeolite. Hence the system is compatible with static, intermittent and continuous arrangements and with equilibrium establishment by diffusion, mixing or forced streaming. Furthermore, as the zeolite has fairly high agent content and microbes tend to grow preferentially on surfaces, rather than in the fluid bulk, the charged zeolite has excellent barrier properties in its neighborhood, preventing infection at its surfaces, through its pores and at possible fissures, imperfections and dead spaces. The barrier properties can be employed to position the zeolite where the infection risks are highest, e.g. between the fluid and an opening to the surrounding, either as an additional precaution or as a measure to reduce the need for agent in the main volume of the fluid. Nothing prevents that the system of the invention is combined with a final filtering step in which the already low agent content in the fluid is further reduced. This can be done either to minimize exposure or to recover the agent for destruction or for feedback of the agent to the zeolite to create a static antiseptic system allowing continuous fluid treatment. The charged zeolites of the present system allow reversible controlled release of the agents both when the fluid is a gas an when the fluid is a liquid and the combined flexibility of the system permits the invention to be adapted for numerous different applications to be exemplified.

Further objects and advantages of the invention will be evident from the detailed description below.

### DETAILED DESCRIPTION

### Definitions

As used herein “system” shall be understood to refer to the principles of the invention generally, whether described, claimed, exemplified or implemented as one or more devices/arrangements, methods, uses or combinations thereof.

In the absence of explicit statements or obvious conditions to the contrary, as used herein expressions like “comprising”, “including”, “having”, “with” and similar terminology shall not be understood to be exclusively restricted to recited device elements, composition compounds/components or method steps but shall be understood to allow for the presence of further elements, compounds/components and steps as well. It shall be understood to cover any device element in integral, subdivided or aggregate forms and expressions like “conected”, “attached”, “arranged”, “applied”, “between” and similar terminology shall not be understood to cover exclusively direct contact between the recited elements but shall be understood to allow for the presence of one or several intervening elements or structures. The same applies for similar expressions when used for description of forces and actions. Similarly, in the absence of explicit statements or obvious conditions to the contrary, such expressions shall be understood to include composition compounds/components in any physical or chemical aggregation or mixture, with possible intervening compounds/components, or state of aggregation as well as method steps in any time sequence.

Also, in the present context “microbes” shall be understood to mean any organism able to survive as single cells in the presence of nutrients or a host organism, e.g. protozoa such as amoebas etc. More narrowly the concept shall have the common meaning of “microorganisms”, i.e. bacteria and fungi of mould or yeast type.

For the purposes of the present invention, in the present context expressions like “antiseptic conditions”, “antiseptic concentration”, “antiseptic level”, antiseptically effective” and similar terminology shall be understood to refer to conditions adapted and suitable for at least retarding growth of, preferably stopping growth of and most preferably killing at least one living microbe, if and when in contact with any component able to act as a nutrient for the microbe, although the expressions shall not be understood to require presence of such a nutrient. Nor shall the expressions be understood to require presence of microbes but include precautionary situations as well. The expressions shall, however, be understood to exclude situations and conditions where the contact or residence time is insufficient for any significant action between the antiseptic and the microbe. The system antiseptic action sufficiency against a single microbe shall be seen in light of the possibility according to the invention to use the charged zeolite as buffer, to release additional antiseptic agent and restore fluid content of agent when the microbe absorbs or otherwise interacts with the agent, and shall not exclude the option that the fluid has an amount or concentration sufficient for being active against more than one or against numerous microbes. Certainly the agent amounts necessary may vary for different agent types, e.g. low for antibiotics having precise metabolic action and high for agents of more bold oxidizing or poisonous action. Accordingly conditions commonly referred to as for example bactericide and bacteristatic are included in the meaning of the expressions.

### The Zeolite

Zeolites can in general be described as crystal frameworks of aluminum silicates or tectosilicates, which can be characterized by their chemical composition and crystal structure.

The chemical composition is generally expressed in terms of the Si/Al ratio. High silica zeolites carry less framework charge and are commonly referred to as hydrophobic. The opposite holds true for high alumina zeolites, which are labeled hydrophilic. Zeolites suitable for the present purposes can be said to have the general structure formula (AlO2)x(SiO2)y wherein the ratio y/x can have different values, to be described. In this zeolitic framework other ions, like P, B, Fe, Ga, Ge etc. may be substituted for Al and Si to a certain degree and can also be used for the purposes of the invention. To this zeolitic framework is bound a cation to each Al atom, or other atom of maximum valence less than four, and a anion to each atom with maximum valence of more than four. Zeolites may contain more or less water.

The crystal lattice provides a pore system where the pores are highly uniform in size although the size is somewhat different between different zeolite types. In general the pores comprises a main cavity and entrance openings. The size of the main cavity varies between about 3 to 11 Å in diameter between different zeolites and the entrances may be about 1 to 3 Å smaller. These uniform pores are responsible for the high and selective absorbency of the zeolites and will be referred to as micro-pores herein. The zeolite crystal may in addition have micro-fissures, resulting from their chemical and physical manufacturing and treatment history, which fissures are not uniform and for the present purposes it is preferred to use zeolites with low or no fissures. Finally, zeolite crystals are normally of limited size, forming a powder or particulate mass. Such a mass can be used as such, e.g. to be suspended in a fluid for absorption. By sintering or by adding a gluing component, e.g. bentonite, talc or phosphate glass, the mass can be consolidated into forms of any shape and size. In doing so macro-pores may be left between the particles. For both unconsolidated powder and consolidated shapes macro-pores between the crystal particle will be referred to as bulk porosity and expressed as the total macro-pore volume to total bulk volume of the zeolite mass or shape respectively. A bulk porosity may serve the purpose of allowing the fluid to enter the macro-pores and contact the individual particle and even allowing the fluid to pass through the zeolite mass or shape. A suitable bulk porosity for such purposes can be at least 20 percent, preferably at least 30 percent and most preferably at least 35 percent and for among others stability reasons the bulk porosity may be at most 90 percent, preferably at most 80 percent and most preferably at most 75 percent. It is certainly possible to use, alternatively or in combination, other known means than pore porosity for creating permeability and/or increasing contact surface between fluid and zeolite, e.g. thin channels through a zeolite bed or body, layers of zeolite etc. For the present purposes zeolites having y/x ratios of 15 and below will be regarded as hydrophilic whereas zeolites with y/x ratios higher than 15 will be regarded as hydrophobic. Both types can be used for the objects of the present invention. Hydrophilic zeolites may for example be used when the antiseptic agent is also hydrophilic. If the agent is ionic it is also possible to utilize the well-known possibility of affecting its affinity to the zeolite by change of the surrounding ionic properties, e.g. pH, lowering the affinity in environments where the agent becomes non-ionic. This may be of interest to fine-tune the release of agent, making self-regulating systems or to make the same charged zeolite useful for the requirements of different fluids. Hydrophilic zeolites may have y/x ratios lower than 15, e.g. lower than 5 and even lower than 1. For reasons indicated it is often preferred to use hydrophobic zeolites, also referred to as de-aluminized or ultra-stable zeolites, e.g. due to their high stability, the possibility to use also the large class of hydrophobic antiseptic agents and non-ionic agents, the latter giving stable affinity properties independent of fluid ionic properties. The hydrophobic zeolites preferably have y/x ratios higher than 100, more preferably higher than 200 and most preferably higher than 1000. Suitable zeolite types may be e.g. silicalite, mordenit and especially zeolite Y. With regard to micro-pore sizes, the zeolite Y and mordenite types belong to the largest known today with pore diameters of about 7 Å and 7.5 Å respectively whereas silicalite has two different pore sizes around 5.5 Å. Silicalite and zeolite Y have three-dimensional pore systems whereas mordenite has two-dimensional pore systems and hence somewhat less accessible. In known manners the hydrophobic zeolites can for example be manufactures through direct synthesis (e.g. silicalite) or by post-synthetic manipulations (e.g. mordenite, zeolite Y), for example by alternating the treatment of zeolite Y with alkali, e.g. ammonia, and acid e.g. hydrochloric acid (USY), or by treatment of zeolite Y with silicon tertrachloride (DAY). Of the post-synthesis manipulated zeolites, the alkali/acid treated have been found faster in its adsorption and release but with a high tendency for adsorption of high-weight proteins on the surface of the particles, whereas the opposite has been found for the silicon tetrachloride treated zeolites.

The zeolite may be used as such or may be treated modify its properties. It is for example possible to coat with e.g. dextran or polyethyleneglycol to reduce the risk for clogging the micro-pore system with larger molecules contained in the fluid.

### The Antiseptic Agent

Generally the antiseptic agents useful for the present purposes should have a size suitable for accommodation in the zeolite micro-pore system. Atoms and atomic ions such as Ag or Cu are easily accommodated and can be used. Preferably the agent comprises molecules, among which a much broader range of suitable agent compounds are available, and such molecules should be possible to accommodate in the micro-pores, at least partially in case of elongated or branched molecules, but preferably the entire molecule should be accommodated. This puts certain limits to the size of suitable molecules and roughly the molecules should have mole weights below 3000, preferably below 2000 and most preferably below 1500. Compounds in these ranges, which can be accommodated in the micro-pores, shall be referred to as “low-weight” whereas larger molecules not able to be accommodated shall be referred to as “high-weight” molecules. Compounds and molecules shall be understood to include aggregates, chelates etc. when sufficiently stable to behave as a unit versus the micropores. A second general requirement is that the agent shall have sufficient affinity to at least one zeolite type to allow absorption in an amount sufficient to give antiseptic conditions in a fluid, at least in a small volume relative to the zeolite, in contact therewith. As indicated, selecting the agent in relation to the zeolite type in general controls this.

Any low-molecular antiseptic agent fulfilling the above requirements can be used according to the invention. Purely oxidizing substances, such as chlorine, hydrogen peroxide etc. can be used as well as purely toxic substances like cyanide although it is preferred to use agents with a more selective action. A suitable class of agents is antibiotics able to inhibit growth or kill microbes at low concentration levels, e.g. Clindamycin or penicillin. Preferred are those not or only moderately toxic against animals or humans, allowing medical use. Antibiotics can be used belonging to different groups with respect to their biological mechanism, such as those affecting synthesis of cell walls, synthesis of proteins, metabolism of folic acid, synthesis of nucleic acid etc. Another suitable class of agents is what broadly is referred to as preservatives, e.g. halogenated compounds like DDT, triclosan etc. or aromates or polyaromates. For medical applications it is preferred to use preservatives approved for such use. Suitable compounds of this type may include benzyl alcohol, bensalconium chloride, cetrimid, chorbutol, chlorohexidine, chlorocresol, hydroxy benzoates, phenyl alcohol, phenoxi alcohol, phenyl mercury nitrate, chlororamphenicol etc. Good results have been obtained with phenol and cresol, especially m-cresol.

The above grouping and classification of agents is made for ease of reference only and shall not be considered limiting as long as the functional requirements are fulfilled. It is fully possible to use mixtures and combination of agents within or between the classes and within or between the hydrophilic and hydrophobic types.

### The Charged Zeolite

The zeolite can be charged with the agent by any known method, e.g. by being contacted with the agent in pure form or in mixture, suspension, emulsion etc. of a media in liquid or gaseous form. The contacting procedure can take place for example by letting the zeolite and agent reside in contact, being agitated together or by passing the agent past or through the zeolite, the latter allowing for a gradient of the agent in the zeolite to be created. Contacting can take place at different temperatures, e.g. elevated temperature to speed up the procedure. The zeolite can be submitted to various after-treatments, e.g. a sterilizing operation based on irradiation, chemical treatment, heating etc., a drying step to give a stable semi-manufacture item for storage or pre-charging to a device before contact with the fluid or an additive treatment step to modify its properties. All these operations are facilitated by the stability properties of the zeolites. Some losses of agent may occur at for example on heat or vacuum treatment, which should be accounted for, e.g. by adding a compensating amount of agent before or after such steps. For example, syringes pre-filled with preparations may need a sterilizing step and if a zeolite is present it will be subjected to the same treatment. Similarly some preparations are subjected to a lyophilization or freeze-drying step under vacuum and any zeolite present can be subjected to the same treatment. During such steps some agent may be lost from the charged zeolite but can be compensated by a corresponding initial over-charging. Also, it has sometimes been observed, especially at a high degree of charging, that some agent becomes absorbed more loosely than the main part of the agent, perhaps due to some absorption outside the micro-pores for example on exterior surfaces or in micro-fissures. It is preferred to avoid inconsistency introduced by such factors, e.g. by subjecting the charged zeolite to a short washing or eluation step to remove to loosely bound agent or by inserting a non-charged or less charged zeolite downstream of the charged zeolite, whereby the first released agent will be captured in the micro-pores of the downstream zeolite until it will have the same saturation degree as the main charged zeolite.

The minimum requirement on the agent and zeolite in the charged zeolite is that a small volume of the fluid, for which the charged zeolite is intended, when contacted with the charged zeolite will reach a minimum antiseptic level of the agent in the fluid, after that the concentration or distribution of the agent between the fluid and the zeolite substantially has reached an equilibrium. These minimum conditions may be useful, for example when in contact with air or a no more than moistened charged zeolite is used as an antiseptic barrier. In general it is preferred to have more than minimum conditions, e.g. to provide for the presence of agent amounts for antiseptic levels in larger volumes of fluid, for example pre-determined volumes surrounding the zeolite or passed through the zeolite, e.g. to suffice for one or more doses of a preparation. These larger agent amounts can be provided either by increasing the volume of zeolite, having low concentration of agent, or preferably by increasing the concentration of agent in the zeolite, e.g. to minimize the amount of zeolite necessary. For very large, or unlimited, volumes of fluid it is preferred to maintain minimum or buffer levels in the zeolite lite by continuous or batch replenishment of the agent in the zeolite, either by adding new agent or by separating out agent form already treated fluid and feeding it back to the zeolite, directly or to the fluid to be contacted with the zeolite. It is also preferred to increase the amount of agent in the zeolite above the minimum requirement to a buffer level in the zeolite, i.e. to levels securing that the agent is present in amounts sufficient for providing safety margin over minimum microbial exposure and preferably to levels sufficient for worst case exposure for the intended use. As indicated, this can be done according to the invention without the sacrifice of any significant increase of agent concentration in the fluid, since the equilibrium between zeolite and fluid will allow repeated and consumption controlled restoration of antiseptic levels in the fluid to about the same agent concentration. For best performance it is preferred that the agent has a fairly high affinity to the zeolite type selected. The affinity shall here be expressed as the amount of agent in the zeolite, weight percent agent in zeolite, at the maximum saturation degree, i.e. the saturation degree obtained with the zeolite in contact with pure agent after sufficient contact time for stabilization. Expressed in this way the maximum saturation degree can be at least 10%, preferably at least 25% and most preferably at least 50%. The maximum saturation degree should be regarded as a gauge value only and shall not necessarily be the degree to which the zeolite is charged for use. When a zeolite is charged to a highly saturated degree, and provided no substantial column effect is present at contact to be described, it will release substantially higher amounts initially than later when the saturation degree has been lowered. The fall off of concentration in the fluid, at continuous or repeated contact with the fluid, is quite rapid and can be approximated with an inverted linear, polynomial or exponential function. High saturation degrees can be used if this concentration pattern is desired, e.g. for a high cleaning burst follow by lower maintaining concentrations. However, for many purposes it is desirable to have substantially constant concentration of the agent in the fluid after contact with the charged zeolite, e.g. at continuous fluid contact of larger fluid volumes or repeated dosing. In order to obtain this, the agent charged to the zeolite should be lower than at the maximum saturation degree and preferably so much lower that the fall off curve has reached an approximately constant behavior. As indication of such saturation degrees may be said that the agent amount in the zeolite should be less than 50% of the amount corresponding to the maximum saturation degree, preferably less than 30% and most preferably less than 10%. For agent and zeolite combinations of high affinity the amounts can be still lower. In general this ratio of actual agent amount to the agent amount corresponding to maximum saturation degree is higher than 0.01%, preferably higher than 0.1% and most preferably higher than 1%. When a column effect is present at the contacting these conditions are less stringent. Provided the charged column is long enough to provide substantially equilibrium agent concentration in the fluid after only partial passage of the column no changes in fluid or zeolite agent amounts will take place during passage of the last parts of the column and the conditions at exit will be highly constant. A parameter of great concern for the present purposes is the concentration of agent in the fluid in equilibrium with the charged zeolite. This concentration can be lower than when not utilizing the principles of the invention, e.g. lower than 0.25 times such a concentration, preferably less than 0.1 times and most preferably less than 0.05 times the concentration allowed in a given application. Absolute concentration values are difficult to give due to e.g. the different antiseptic efficacy of different classes of agents and differences between technical applications and treatment applications respectively. A typical demanding application is liquids for body injection where a typical authority approved concentration of preservatives is about 2-3 mg/ml where the above said general reductions are possible. Values down to 0.01 mg/ml have been proved still efficient. Similarly absolute charging values are difficult to give although as an indication charging rations w/w of agent to zeolite in general is higher than 0.1 percent, preferably more than 1 percent and most preferably more than 5 percent or even more than 10 percent or more than 20 percent.

In view of the variations possible the above given considerations shall be regarded mainly as guidelines. Adaptations towards higher agent amounts may be needed where the contact time between zeolite and fluid is too short for equilibrium or where the agent and zeolite combination moves slowly towards equilibrium. Temperature should also be considered. In general the agent equilibrium concentration in a fluid in contact with a given charged zeolite will increase with temperature. Microbes may also be more active at higher temperatures. Although most often the operation temperatures are dictated by use constraints, the fluid/zeolite combination may be somewhat self-regulating.

### The Fluid

The present system can be used for control of microbial growth in broad ranges of fluids. The fluid may be a pure substance or a mixture of components of the same or different states of aggregation. A gas as continuous phase may contain liquid drops or solid particles. A liquid as continuous phase may contain particles of solids or droplets of liquids and the mixtures may be suspensions, emulsions etc. The fluids may be simple mixtures, such as air or water solutions or mixtures, or may be complex mixtures of even unknown content, e.g. a contaminated stream or a body fluid. It is preferred to use the system for fluids that not too strongly interfere with the release mechanism described. The fluid should only contain small amounts of compounds or compositions able to precipitate on or adhere to the zeolite to such an extent as to block micro-pores or the macro-pores. Preferably the fluid has a not too high viscosity, e.g. below 10000 cP, preferably below 1000 cP and most preferably below 100 cP. The fluid may contain compounds that compete with the agent for zeolite affinity, e.g. to create a control means for release of varying agent amounts depending on presence or added amounts of such a competing compound. The competing compound may then have an affinity, as defined, to the zeolite similar to the agent or even larger than the agent, e.g. more than 2 times, preferably more than 10 times and most preferably more than 20 times the affinity of the agent. Such compound shall also be understood to include typical eluating media, e.g. media that affects the absorbed agent to a state of less affinity to the zeolite, e.g. by reducing or increasing its ionic character for hydrophilic and hydrophobic zeolites respectively. However, for reasons outlined it is often preferred that the controlled release of agent means a substantially constant level of agent in the fluid. For such purposes it is preferred that the liquid have only small amounts of compounds that compete with the agent for zeolite affinity, i.e. compound that are both low-molecular in the above discussed sense and have high affinity to the zeolite. The fluid may contain large amounts of low-molecular compounds provided these have lower affinity to the zeolite than the agent, e.g. an affinity, as defined, of at most 0.5 times that of the agent, preferably at most 0.1 times and most preferably at most 0.05 times that of the agent. Such compounds may be air or water molecules, having low affinity to hydrophobic zeolites, or small non-ionic compounds, having low affinity to hydrophilic zeolites. Similarly the fluid may well contain large amounts of high-molecular compounds, as defined, since these do not tend to be absorbed by the zeolite independent of their hydrophobic or hydrophilic properties respectively. It is often preferred to use the invention in connection with fluids containing such compounds, e.g. for medical preparations where the compounds can be for example proteins, polypeptides, carbohydrate compounds, nucleic acid sequences etc., hereby utilizing the zeolite property of not absorbing these kind of large molecules. As a rough indication of “large amounts” in the above sense can be above 0.01 mg/ml, preferably above 0.1 and most preferably above 1 mg/ml. Similarly “small amounts” may refer to less than these values. The advantages of the present invention are especially pronounced when the fluid contains components serving as nutrients for the microbes and especially when such nutrients are present in large amounts. Further, when using the invention it is not necessary to include preservatives in the fluid and it is accordingly preferred that the fluid contains no or only small amounts of preservatives of either the agent or preferably any other preservative.

### Fluid/zeolite Contact

As indicated the fluid and the zeolite can be brought into contact in different ways. The contact can be made by keeping the fluid static with respect to the zeolite, typically relying on a diffusion of agent from the zeolite to the fluid, resulting in concentration gradients in at least the fluid before saturation has been achieved throughout the fluid volume. Contacting can also be made by relative movement between the fluid and the zeolite, e.g. by agitating the fluid or forcing it to stream past or through the zeolite, typically resulting in less concentration gradients in the fluid. The zeolite may be in particulate form suspended in the fluid, typically giving small agent concentration gradients in the zeolite. The zeolite may be present in the form of a coating on a surface in contact with the fluid or a bed or column past or through which the fluid is moved, typically resulting in a concentration gradient for the agent in the zeolite, with increasing amounts when moving from the upstream to the downstream side of the zeolite. A bed or column also has the advantages of facilitating contact with all fluid, providing an additional barrier effect and providing bulk porosity or interstices for the fluid to occupy and improving contact. A column of not insignificant length may also serve the purpose of maintaining highly constant agent concentrations at column exit by saturating the fluid already at the entrance or intermediate parts of the column while maintaining substantially constant agent amounts in the zeolite at the exit. For all arrangements the agent concentration in the fluid typically increases at contact with the zeolite, at least initially and provided that equilibrium has not already been reached by earlier contact or pre-charging. It is advantageous to reach equilibrium or almost equilibrium and this may be obtained, e.g. when storing the fluid for sufficient time in contact with the charged zeolite. However, it is not always necessary to reach such equilibrium but may be sufficient to reach an acceptable antiseptic level concentration, e.g. for avoiding too long contact times, in relation to the speed of the agent/zeolite combination, for example in a system with streaming fluid. Still it may be possible and preferable to obtain steady state conditions at a suitable antiseptic level. Generally the contact may take place by a batch operation, by intermittent operation or by continuous contact. In batch or intermittent operations there are some advantages, at least for smaller fluid volumes, to use a zeolite bed or column having sufficient bulk porosity to accommodate much, preferably most and most preferably all of the fluid in the macro-pores, i.e. so that the bed contains the fluid volume. Among others this optimizes contact conditions during the available residence time. For intermittent operations it is preferred that fluid volume accommodated corresponds at least to the volume of the doses to be repeated or, in case of varying doses, to the largest dose considered.

### Arrangements

As said the charged zeolite may have utility as such, e.g. due to its excellent barrier properties, and may then just be adapted to the specific application where it is intended to be applied as barrier, e.g. formed or inserted into a sealing part or being designed as a patch for attachment on a part to be protected, e.g. a wound. A preferred arrangement is to have at least a chamber for the fluid in combination with the zeolite. A chamber may be open, e.g. an open vessel where, or closed, like a vial or other enclosure, or being part of a conduit, like in a transport channel for the fluid up to the zeolite. In such arrangements the charged zeolite may serve to maintain the content sterile, to act as a barrier e.g. over an opening to prevent contamination or to raise the agent concentration in the fluid when the fluid is passed from the chamber past or preferably through the zeolite out from the chamber. Another preferred arrangement is to have a first, upstream chamber, a second, downstream, chamber and the charged zeolite arranged so as to allow the fluid to come into contact with the zeolite at least when passing from the upstream chamber to the downstream chamber in such a way that the fluid content of agent increases. Such an arrangement may for example be a growth control part of a fluid transport channel for any purpose or be part of a fluid delivery arrangement. For reasons given it is preferred to apply the zeolite in the form of a bed or column allowing passage of the fluid through the bed.

Although the arrangements are intended for interaction with the fluid, the arrangements shall be regarded as a part of the present system when useful or adapted for the purposes of the invention. The arrangements may be useful also when the fluid does not necessarily come into contact with the zeolite, for example when the zeolite is used as a precautionary measure, e.g. to be activated in case a sealing is inadvertently broken or becomes defect. In some instances such limitations are intentionally introduced, e.g. when valves, rupturable, pierceable or removable membranes or other sealings are inserted in the arrangement to allow creation of fluid contact at a controlled moment, e.g. in connection with activation or opening of a pre-filled device.

The general arrangements outlined can be adapted for numerous applications and the specific applications may affect the arrangement details. In addition to the uses already indicated further applications will be exemplified in connection with the Figures.

### SUMMARY OF DRAWINGS

FIG. 1 illustrates schematically in box form the contemplated equilibrium system of the invention under idealized conditions.

FIG. 2 illustrates schematically an eluation curve for a zeolite initially charged to a high saturation degree.

FIG. 3 illustrates schematically a zeolite bed in the form of a column of sufficient length to allow for gradients of agent amounts in the zeolite to form.

FIG. 4 illustrates schematically various zeolite arrangements in connection with a single chamber.

FIG. 5 illustrates schematically various zeolite arrangements in connection with an upstream chamber and a downstream chamber.

FIG. 6 illustrates a diagram in relation to Example 1.

FIG. 7 illustrates a diagram in relation to Example 2.

FIG. 8 illustrates a diagram in relation to Example 5.

FIG. 9 illustrates a diagram in relation to Example 6.

FIG. 10 illustrates a diagram in relation to Example 7.

### DESCRIPTION OF DRAWINGS

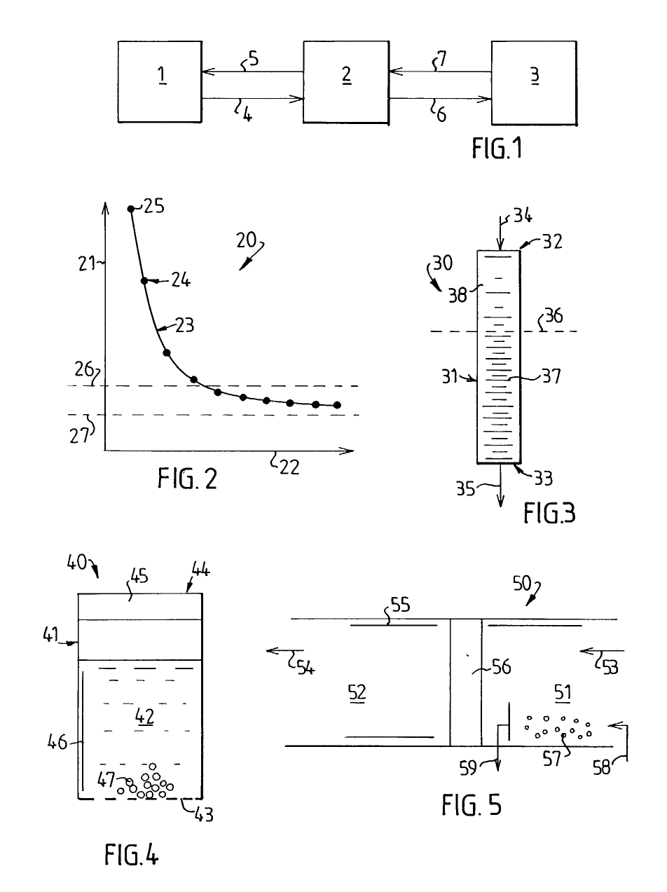

FIG. 1 illustrates schematically in box form the contemplated equilibrium system of the invention under idealized conditions. The left box illustrates the charged zeolite 1, the middle box illustrates the fluid 2 being in contact with the zeolite 1 and the right box illustrates microbes 3 present in the fluid. The zeolite and the fluid are in reversible equilibrium with each other, which is illustrated with arrows 4 and 5. Similarly the microbes and the fluid are in reversible equilibrium, which is illustrated by arrows 6 and 7. Arrow 4 indicates the flow of agent from the zeolite to the fluid, e.g. initially when the fluid is under-balanced with agent or in any situation where the fluid becomes under-saturated for example in connection with consumption of agent by the microbes or addition of new fluid. Arrow 5 indicates the opposite flow of agent from the fluid to the zeolite if or when the fluid becomes over-saturated with agent, for example if liquid fluid is evaporated or if the microbes release the agent when destroyed. In most uses the back-flow of agent according to arrow 5 is less important than the arrow 4 flow. Arrow 6 indicates the flow of agent from the fluid to the microbes, here assumed to be present. Consumption of agent according to arrow 6 may result in a lowering of agent concentration in the fluid 2 and a restoring flow of agent from the zeolite 1 to the fluid 2 according to arrow 4. Arrow 7 indicates the theoretical possibility that agent is released to the fluid 2 from the microbes, e.g. in connection with their death and decay, which in turn may result in a corresponding equalizing flow back to the zeolite according to arrow 5. Arrow 7 flow is highly dependent on the mechanism between agent and the microbes and is not necessarily always present. It may also be non-existing if killed microbes are removed from the system. It should be noted that box 3 could also be said to illustrate any component or disturbance, other than microbes, acting to destroy or consume agent from the fluid 2, e.g. an impurity in the fluid or a compound in a complex fluid preparation to which the agent may be bound, absorbed etc. Accordingly it is clear that the system illustrated is highly resilient against any form of disturbances or any form of agent consumption. For the purposes of the invention it is desirable to keep the concentration of agent in the fluid 2 to an antiseptic level but otherwise quite low, allowing the fluid to be much less harmful than if the buffering capability of the charged zeolite would not be present. The speed with which the system moves towards a new equilibrium at a disturbance may vary. In general terms can be said that the equilibrium between the zeolite and the fluid is quite fast at their interface but slower where diffusion is necessary for fluid volumes not in contact with the zeolite. However, agitation or fluid flow may be used to remedy diffusion delays. The equilibrium between the fluid and the microbes is highly dependent on the antiseptic mechanism but may be slower, although this is typically less important for the system overall performance.

FIG. 2 illustrates schematically an eluation curve 20 for a zeolite initially charged to a high saturation degree. The vertical axis 21 represents the concentration of agent in the fluid having been in contact with the zeolite and the horizontal axis 22 represents the volume of fluid or number of re-suspensions. The solid curve 23 can be said to represent the pattern obtained when contacting the fluid continuously with the zeolite and the discrete values 24 can be said to represent the concentrations achieved at repeated batch contacting. The curve illustrates that an initially high concentration 25 is obtained in the fluid, which concentration rapidly falls off to a state where further volumes or further re-suspensions give substantially constant concentration of agent in the fluid. Dotted lines 26 and 27 indicate roughly upper and lower charging degrees respectively that can be selected to give substantially constant agent concentrations in the treated fluid. Suitable initial zeolite charge can be obtained by over-charging it followed by eluation or evaporation of agent until the level of line 26 is reached or preferably by initially charging the zeolite to a level corresponding to line 26, e.g. by use of a diluted charging fluid. It should be noted that the curve represents the agent amount in the fluid and not in the zeolite. If the agent has high affinity to the zeolite the zeolite may comprise enormous amounts of the agent in spite of the fact that the concentration in the fluid is low, allowing large volumes of fluid or numerous contact repetitions with substantially constant agent concentration in the outgoing fluid. It should further be noted that the curve illustrated in FIG. 2 is typical for contact patterns where no substantial agent amount variations or gradients results in the zeolite, for example no column effects. Such an equalized state may result e.g. when the zeolite is mixed or agitated randomly with the fluid or when a bed of zeolite is thin or shallow.

FIG. 3 illustrates schematically an arrangement, generally designated 30, with a zeolite bed in the form of a column 31 of sufficient length to allow for gradients of agent amounts in the zeolite to form. If the fluid residence time is sufficient in relation to the speed of the equilibrium system, the fluid will typically become saturated with the agent during passage if an early fraction of the column height after which the fluid will pass the remainder of the column without further exchange of agent between zeolite and fluid, i.e. with both fluid and zeolite remaining unchanged. This will allow highly constant fluid exit concentrations for large volumes of fluid until the column has been depleted of agent to such a degree that passage of the entire column will not any longer give the target concentration. In the Figure the column 31 has an entrance end 32 and an exit end 33. Initially, before contact with any fluid, the column is assumed to have an over its length constant charge of agent. Fluid is then passed from the entrance 32 end of the column, as illustrated by arrow 34, to the exit end 33, as illustrated by arrow 35. After a certain time of operation the conditions illustrated will be reached. Typically a dynamic borderline, illustrated with dotted line 36, will form, separating a lower part 37 of the column, with unaffected agent amounts, from an upper part 38 of the column, with reduced agent amounts. The fluid will reach saturation concentration of the agent during passage of the upper part 38 of the column, between the entrance 32 and the borderline 36, and in this part the column will have a gradient of agent amount increasing from the entrance to the borderline. During passage of the remainder and lower part 37 of the column, from the borderline 36 to the exit 33, no substantial changes will take place either in the fluid or the column and the column will have substantially its initial content of agent and no gradient will form and the fluid will have constant exit concentration. When continuing feeding fluid to the column the borderline 36 will move slowly downwards until it reaches the exit end 33. The entire column will now have a gradient of diminishing agent amounts towards the exit end and the concentration of agent in the fluid will begin to drop. In contrast to the situation described in connection with FIG. 2, it is not any longer necessary to limit the zeolite saturation degree from the beginning to obtain stable exit concentrations. Instead the column effect secures a constant exit concentrations independent of initial saturation degree. Even very high or maximum saturation degrees can be used, although in that case a zeolite and agent combination with stronger agent bonding to the zeolite can be selected if exit concentrations similar to that of the FIG. 2 system shall be targeted.

FIG. 4 illustrates schematically various zeolite arrangements, generally designated 40, in connection with a single chamber 41, here comprising a liquid fluid 42. The chamber may be closed or may be a part of a conduit or larger system as indicated by dotted line 43.

The chamber is also shown with an opening 44, which opening is sealed with a charged zeolite in the form of a fixed disc 45, e.g. of particulate zeolite arranged between retaining sieves or preferably a sintered self-supporting body. The charged zeolite in the opening 44 is here exposed to the surrounding and may act as an antiseptic barrier against microbial infection of the chamber. In the position shown in the Figure the liquid 42 is not in contact with the zeolite disc 45 and the zeolite can be regarded as a safety mean and the liquid need not necessarily have an antiseptic level of agent. Still the opening may allow access to the liquid, e.g. pouring or forcing it through the porous disc. Also illustrated is another fixed arrangement of the zeolite as a coating 46 on the chamber interior surface. Due to the possibility of charging a zeolite with high amounts of agent to give low concentrations in the fluid it is often possible to use fairly small amounts of charged zeolite and a partial coating can be fully sufficient. Further illustrated is non-fixed amount of zeolite 47 in particulate or powder form, which can be residing in the chamber or agitated to be suspended. The arrangements shown can be used for protection of chambers for various purposes or sizes, e.g. enclosures for fluids to large manufacturing plants for example in connection with shut down and preservation of the plant.

FIG. 5 illustrates schematically various zeolite arrangements, generally designated 50, in connection with an upstream chamber 51 and a downstream chamber 52 for a streaming fluid fed to the upstream chamber, as illustrated by arrow 53, and extracted from the down-stream chamber, as illustrated by arrow 54. Between the chambers are arranged a charged zeolite, either as coatings 55 on the walls or preferably by a bed 56 thought which the fluid is forced to pass. Also schematically illustrated is the possibility to suspend a zeolite in particulate or powder form 57 in the incoming fluid stream 53, e.g. through a zeolite feeding line 58, and collecting it further downstream with for example a filter for possible removal, e.g. through a zeolite extraction line 59, possibly for repeated feeding to the zeolite feeding line 58. Such an arrangement gives an additional control degree in that the charged zeolite fed into line 58 can be externally manipulated to optimize its condition, e.g. with agent re-charging to minimize the amount of zeolite or to adapt its agent amount or agent type to varying conditions in the incoming fluid 53. Also the arrangement shown in this figure can be adapted for numerous applications with streaming fluids and arrangement sizes, e.g. from a small syringe to a large manufacturing or fluid cleaning plant.

### EXAMPLE 1

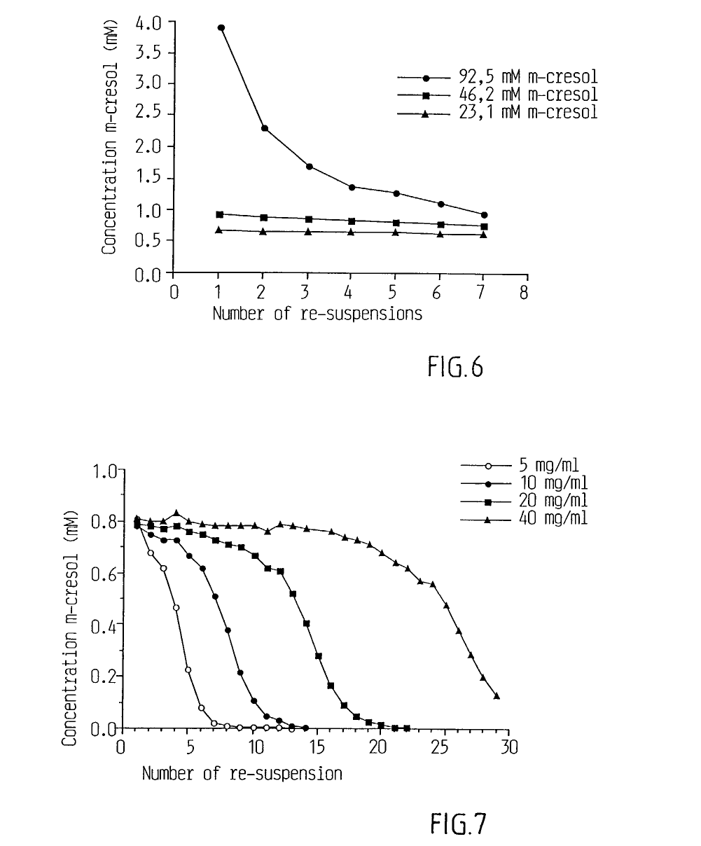

This example illustrates release of m-cresol from zeolite with adsorbed m-cresol and is described with reference to the diagram in FIG. 6.

After incubation with varying concentrations of m-cresol the zeolite was allowed to sediment and the supernatant was removed. Then the zeolite was suspended in PBS (phosphate buffer salin) so that an amount corresponding to 20 mg dry zeolite per ml was suspended and after 30-60 minutes of incubation on a rocker table the concentration of cresol in the supernatant was measured by absorption at 276 nm, after which the zeolite was suspended in a new portion of PBS. The concentration of cresol was again determined in the supernatant and the zeolite was again suspended in PBS.

### EXAMPLE 2

This example illustrates release of m-cresol from zeolite containing adsorbed m-cresol and is described with reference to the diagram in FIG. 7.

After incubation with 23.1 mM m-cresol the zeolite was dried on glass filter and the zeolite was suspended repeatedly in PBS amounts corresponding to 5-20 mg dry zeolite per ml. Between each suspension the zeolite was incubated on a rocker table. The concentration of cresol in the supernatants were analyzed by Abs 276 nm.

### EXAMPLE 3

This example illustrates growth inhibition of Staphylococcus aureus (ATCC 6538), FU ml-1) after incubation with m-cresol adsorbed on zeolite. Test according to Eur. Pharm. nd Ed. VIII 14 (1992). Efficacy of antimicrobial preservation.

Ultra-stable zeolite Y (USY, 63-125 μm particles) was incubated with m-cresol 5 g/ml after which the zeolite was sucked dry and further dried in warm closet overnight. Zeolite with adsorbed m-cresol was then suspended in PBS and was incubated with Staphylococcus aureus at 37° C. The concentration of m-cresol in the solution after the suspension of zeolite was 0.14 mg/ml.

| Incubation time | Blank^{a} | Zeolite 50 mg/ml | Free m-cresol^{b} |
|---|---|---|---|
| (h) | CFU/ml | CFU/ml | CFU/ml |
| 0 | 5.8 × 10⁶ | 5.8 × 10⁶ | 5.9 × 10⁶ |
| 2 | 5.4 × 10⁶ | 132 | 1.2 × 10² |
| 4 | 6.3 × 10⁶ | 25 | 24 |
| 8 | 7.9 × 10⁶ | 13 | 3 |
| 24 | 5.5 × 10⁶ | 6 | 0 |
| 48 | 9.1 × 10⁶ | 0 | 0 |

^{a}Blank: zeolite without any adsorbed m-cresol (25 mg/ml).
^{b}5 mg m-cresol/ml dissolved in water as positive control

### EXAMPLE 4

This example illustrates growth inhibition of Staphylococcus aureus (ATCC 6538), CFU ml-1) after treatment with m-cresol adsorbed to zeolite.

Test according to Eur. Pharm. 2nd Ed. VIII 14 (1992). Efficacy of antimicrobial preservation. Dealuminated zeolite Y (DAY, 63-125 μm particles) was incubated with m-cresol 5 mg/ml after which the zeolite was sucked dry and further driedn in warm closet overnight. The zeolite was then suspended in phosphate buffer (0.3336 mg/ml mono-sodium phosphate, 0.7064 mg/ml di-sodium phosphate) with 0.2 mg glycine/ml and 41 mg mannitol/ml with and without addition of growth hormone (GH) after which the zeolite was incubated with Staphylococcus aureus at 37° C.

| | | | 30 mg/ml | 30 mg/ml | 50 mg/ml |
|---|---|---|---|---|---|
| Incub. | | 30 mg/ml | z. + GH | z. + GH | z. + GH |
| Time | Blank^{a} | zeolite (z.) | 5 mg/ml | 1 mg/ml | 5 mg/ml |
| (h) | CFU/ml | CFU/ml | CFU/ml | CFU/ml | CFU/ml |
| 0 | 6.2 × 10⁶ | 6.4 × 10⁶ | 6.4 × 106 | 6.4 × 106 | 6.4 × 106 |
| 1 | 6.3 × 10⁶ | 5.9 × 104 | 8.1 × 104 | 7.2 × 104 | 8.7 × 103 |
| 4 | 6.1 × 10⁶ | 3.3 × 104 | 3.2 × 104 | 6.9 × 104 | 3.3 × 103 |
| 8 | 5.8 × 10⁶ | 1.2 × 104 | 8.5 × 103 | 5.7 × 104 | 1.6 × 103 |
| 24 | 4.9 × 10⁶ | 7.9 × 103 | 1.8 × 103 | 2.2 × 104 | 7.4 × 102 |
| 48 | 4.7 × 10⁶ | 3.6 × 103 | 1.6 × 102 | 6.7 × 103 | 73 |

^{a}Blank: enbart buffert fosfat, glycin, mannitol

### EXAMPLE 5

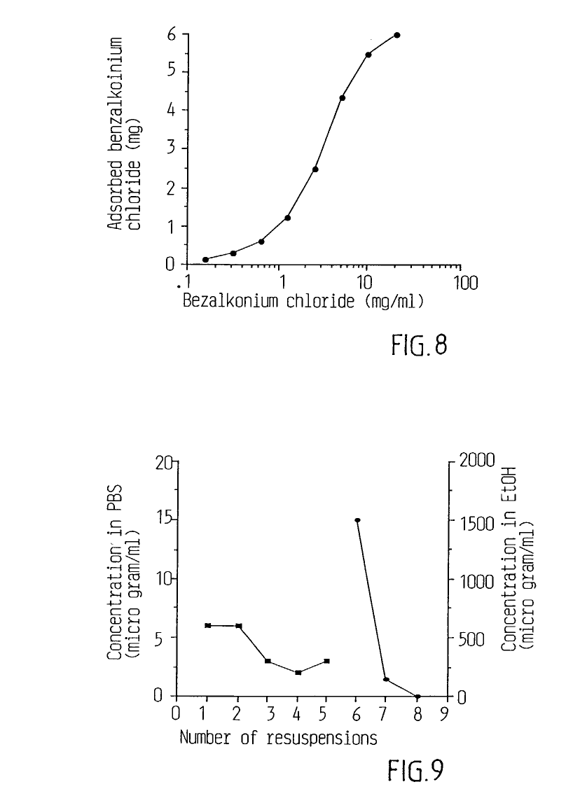

This example illustrates adsorption of benzalkonium chloride to ultra-stable zeolite Y (USY) and is described with reference to the diagram in FIG. 8.

Benzalkonium chloride (156-20 mg/ml) was incubated 60 minutes on a rocker table with 25 mg ultra-stable zeolite Y per ml (USY particles 63-125 μm). The amount of free benzalkonium chloride was determined by absorbency 263 nm and the amount of benzalkonium chloride adsorbed to the zeolite was calculated.

### EXAMPLE 6

This example illustrates release of benzalkonium chloride from ultra-stable zeolite Y (USY) and is described with reference to the diagram in FIG. 9.

Ultra-stable zeolite Y (USY particles 63-125 μm) was incubated with benzalkonium chloride 5 mg/ml after which the zeolite was sucked dry and further dried in warm closet overnight. Zeolite with adsorbed benzalkonium chloride was repeatedly suspended to 20 mg/ml, first in PBS (▪) and then in 95% ethanol (●). The concentration of benzalkonium chloride in the solutions was determined by absorbency at 263 nm.

### EXAMPLE 7

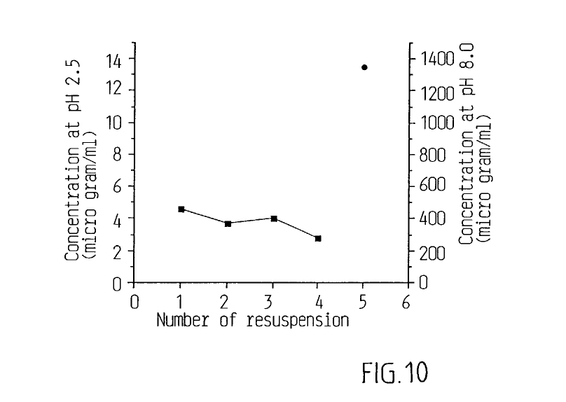

This example illustrates release of cephalothin from ultra-stable zeolite Y (USY) and is described with reference to the diagram in FIG. 10.

Ultra-stable zeolite Y (USY) was incubated with the antibiotic cephalothin 5 mg/ml after which the zeolite was sucked dry and further dried in warm closet. Zeolite with adsorbed cephalothin was suspended repeatedly to 20 mg/ml in 10 mM glycin pH 2.5 (▪) and the concentration of cephalothin in the solutions was determined by absorbency measurement at 260 nm. Then the zeolite was suspended in 10 mM phosphate buffer pH 8.0 (●). The change in pH makes the cephalothin de-protonized and the charge introduced results in an increased release of cephalothin from the zeolite.

### EXAMPLE 8

This example illustrates adsorption and release of various antibiotics. Ultra-stable zeolite Y (USY) was incubated with various antibiotics and the amounts absorbed to the zeolite were calculated. Then the zeolite was dried and suspended to 20 mg/ml in buffer and the concentrations of released antibiotics in the solutions were determined.

| | Amount adsorbed | Amount antibiotic | Release |
|---|---|---|---|
| | antibiotic/ | in 20 mg | concentration |
| Antibiotic | mg zeolite | zeolite/ml | of antibiotic |
| Ampicilin | 0.14 mg | 2.8 mg/ml | 400 μg/ml |
| Cephalothin | 0.21 mg | 4.2 mg/ml | 4.6 μg/ml |
| Chloramphemicol | 0.125 mg | 2.5 mg/ml | 7 μg/ml |
| Gentamycin | 0.18 mg | 3.6 mg/ml | inte gjord |
| Streptomycin | 0.48 mg | 9.6 mg/ml | 4800 μg/ml |
| Tetracyclin | 0.09 mg | 1.8 mg/ml | 220 μg/ml |

### EXAMPLE 9

Dealuminated zeolite (DAY; 63-125 microns) was charged with m-cresol (10 mg/ml) at a zeolite content of 40 mg DAY per ml m-cresol solution. An E. Coli (CU1867, ATCC# 47092) suspension was mixed with uncharged zeolite and zeolite charged with m-cresol and was allowed to sediment for 5 minutes. The supernatant was removed from the zeolite sediment and the zeolite with remaining confined bacterial suspension (8×10⁶ colony forming units, CFU) was incubated at room temperature. After 18 hours the sediment was re-suspended and the number of CFU was determined after coating on LB-agar. The test were made both in buffer (0.3336 mg/ml mono- sodium phosphate, 0.7064 mg/ml di-sodium phosphate, 2 mg/ml glycin and 41 mg/ml mannitol) and in buffer containing growth hormone (GH, 5.5 mg/ml). The distribution from two tests are given below in CFU.

| | Zeolite Y | Control zeolite Y |
|---|---|---|
| | containing cresol | (without m.cresol) |
| Zeolite in | 2.2 × 10⁶ +/− 0.1 × 10⁶ | 67.5 × 10⁶ +/− 38.9 × 10⁶ |
| buffer | | |
| Zeolite in | 2.1 × 10⁶ +/− 0.4 × 10⁶ | 122.5 × 10⁶ +/− 17.7 × 10⁶ |
| buffer and | | |
| growth | | |
| hormone | | |

### EXAMPLE 10

Dealuminated zeolite (DAY; 63-125 microns) was charged with m-cresol (10 mg/ml) at a zeolite content of 40 mg DAY per ml m-cresol solution. An E. Coli (CU1867, ATCC# 47092) suspension was mixed with uncharged zeolite and zeolite charged with m-cresol and the mixtures were allowed to sediment for 5 minutes. The supernatant was removed from the zeolite sediment and the zeolite with remaining confined bacterial suspension (8×10⁶ colony forming units, CFU) was incubated at room temperature and at +8° C. respectively. The test were made in LB-medium and after 18 hours at-room temperature and after 2.5 days at +8° C. respectively the sediments were re-suspended and the number of CFU was determined after coating on LB-agar. The results are given below in CFU.

| | Zeolite Y | Control zeolite Y |
|---|---|---|
| | containing cresol | (without m.cresol) |
| Zeolite at room temp. | 4.1 × 10⁴ | 10,000 × 10⁴ |
| Zeolite at 8° C. | 16.3 × 10⁴ | 14.7 × 10⁴ |

1. An arrangement for controlling microbial content or growth in a fluid, comprising a) a zeolite with micro-pores charged with an agent having affinity to the zeolite micro-pores and having antiseptic properties and b) the fluid, the zeolite and the fluid being enclosed by, at least partially in contact, or being arranged for being brought at least partially in contact, characterized in that the fluid contains a therapeutically active compound or composition in a therapeutically effective amount and concentration and that the total amount of agent in the zeolite is larger than an amount corresponding to an antiseptic level for the fluid.

2. The arrangement of claim 1, characterized in that the micro-pores of the zeolite has main cavities of uniform size somewhere between 3 and 11 Å.

3. The arrangement of claim 1, characterized in that the zeolite comprises a powder or particulate mass.

4. The arrangement of claim 1, characterized in that the zeolite comprises a structure of consolidated powder or particulate mass.

5. The arrangement of claim 4, characterized in that the bulk porosity of the structure is at least 20 percent, preferably at least 30 percent and most preferably at least 35 percent and preferably at most 80 percent, preferably at most 70 percent and most preferably at most 65 percent

6. The arrangement of claim 1, characterized in that the zeolite comprises hydrophobic zeolite, the zeolite having the general structure formula (AlO2)x(SiO2)y wherein the ratio y/x is at least 15, preferably more than 100, preferably more than 200 and most preferably more than 1000.

7. The arrangement of claim 1, characterized in that the agent comprises, consists essentially or consists of molecules having mole weights below 3000, preferably below 2000 and most preferably below 1500.

8. The arrangement of claim 1, characterized in that the agent comprises at least one antibiotic.

9. The arrangement of claim 1, characterized in that the agent comprises at least one preservative.

10. The arrangement of claim 9, characterized in that preservative comprises one from a group consisting of benzyl alcohol, bensalconium chloride, cetrimid, chorbutol, chlorohexidine, chlorocresol, hydroxy benzoates, phenyl alcohol, phenoxi alcohol, phenyl mercury nitrate, chlororamphenicol, phenol, cresol, especially m-cresol, and combinations thereof.

11. The arrangement of claim 1, characterized in that the agent affinity to the zeolite, expressed as the w/w amount of agent in the zeolite at the maximum saturation degree, of at least 10%, preferably at least 25% and most preferably at least 50%.

12. The arrangement of claim 1, characterized in that the w/w amount of agent in the zeolite is less than 100% of the amount corresponding to maximum saturation degree, preferably less than 50%, more preferably less than 30% and most preferably less than 10%.

13. The arrangement of claim 1, characterized in that the w/w amount of agent in the zeolite is higher than 0.01% of the amount corresponding to maximum saturation degree, preferably higher than 0.1% and most preferably higher than 1%.

14. The arrangement of claim 1, characterized in that the amount of agent charged to the zeolite is adapted to give release of agent to an antiseptic concentration of the agent in the fluid when in contact with the fluid

15. The arrangement of claim 1, characterized in that the zeolite forms a column of sufficient length to provide substantially equilibrium agent concentration in the fluid after partial passage of the column.

16. The arrangement of claim 1, characterized in that the fluid comprises gas.

17. The arrangement of claim 1, characterized in that the fluid comprises liquid.

18. The arrangement of claim 1, characterized in that the fluid comprises low-weight, as defined, molecules having lower affinity, as defined, to the zeolite than the agent.

19. The arrangement of claim 18, characterized in that the affinity of the low-weight molecules is at most 0.5 times, preferably at most 0.1 times and most preferably 0.05 times that of the agent.

20. The arrangement of claim 1, characterized in that the fluid comprises high-weight, as defined, molecules.

21. The arrangement of claim 20, characterized in that the amount of high-weight molecules is above 0.01 mg/ml, preferably above 0.1 and most preferably above 1 mg/ml.

22. The arrangement of claim 20, characterized in that the high-weight molecules include one selected from a group consisting of proteins, polypeptides, carbohydrates, nucleic acid sequences, lipids or mixtures thereof.

23. The arrangement of claim 20, characterized in that the high-weight molecules includes at least one therapeutically active compound or composition.

24. The arrangement of claim 1, characterized in that the fluid contains nutrient components for microbes.

25. An arrangement for controlling microbial content or growth in a fluid, comprising a) a zeolite with micro-pores charged with an agent having affinity to the zeolite micro-pores and having antiseptic properties and b) the fluid, the zeolite and the fluid being enclosed by, at least partially in contact, or being arranged for being brought at least partially in contact, characterized in that the fluid contains molecules being larger than the micro-pores of the zeolite and having less affinity, as defined, to the zeolite than the agent.

26. The arrangement of claim 25, characterized in that the molecules constitutes or form part of a therapeutically active compound or composition.

27. The arrangement of claim 26, characterized in that the therapeutically active compound or composition is present in a medically active concentration in the fluid.

28. The arrangement of claim 25, characterized in any characteristic of claims 1 to 24.

29. A method for controlling microbial content or growth in a fluid, the fluid containing a therapeutically active compound or composition in a therapeutically effective amount and/or concentration, characterized in the step of contacting at least a part of the fluid with a zeolite with micro-pores, charged with an agent having affinity to the zeolite micro-pores and having antiseptic properties, to release agent from the zeolite and increase the content of agent in at least a part of the fluid.

30. The method of claim 29, characterized in that the contacting step includes the step of keeping the fluid in substantially static relationship with respect to the zeolite.

31. The method of claim 29, characterized in that the contacting step includes the step of moving the fluid with respect to the zeolite.

32. The method of claim 31, characterized in that the moving step includes agitation of the fluid and/or the zeolite.

33. The method of claim 31, characterized in that the moving step includes the step of passing the fluid past the zeolite.

34. The method of claim 31, characterized in that the moving step includes the step of passing the fluid through macro-pores in the zeolite.

35. The method of claim 34, characterized in the step of passing the fluid through a column of the zeolite of sufficient length to give substantially equilibrium concentration of agent in the fluid before or at column exit.

36. The method of claim 29, characterized in the contacting step includes the step of maintaining the fluid in static relationship within macro-pores in the zeolite.

37. The method of claim 36, characterized in that during the static relationship a substantially antiseptic level of agent in the fluid is maintained.

38. The method of claim 29, characterized in the step of increasing the content of agent in the fluid to an antiseptic level in the fluid.

39. The method of claim 29, characterized in the step of increasing the content of agent in the fluid substantially to an equilibrium level with respect to the charged zeolite.

40. The method of claim 29, characterized in that the contacting step includes batch contact between the fluid and the zeolite.

41. The method of claim 29, characterized in that the contacting step includes intermittent contact between more than one dose of the fluid and the zeolite.

42. The method of claim 41, characterized in that the intermittent contact includes the step of maintaining at least a part and preferably all of the fluid dose in static relationship within macro-pores in the zeolite.

43. The method of claim 29, characterized in the contacting step includes continuous contact between the fluid and the zeolite.

44. The method of claim 43, characterized in the step of continuous or batch replenishing the agent in the zeolite.

45. The method of claim 44, characterized in that the replenishment step includes the step of feeding agent to the fluid before contact with the zeolite.

46. The method of claim 44, characterized in that the replenishment step includes the step of separating out agent from the fluid after contact with the zeolite and feeding it to the zeolite.

47. The method of claim 46, characterized in that the separating step includes the step of extracting the agent by contacting the fluid with a second zeolite, charged with no or less agent than the zeolite.

48. The method of claim 29, characterized in any characteristic of the preceding claims.

49. A method for controlling microbial content or growth in a fluid, characterized in the step of contacting at least a part of the fluid with a zeolite with micro-pores, charged with an agent having affinity to the zeolite micro-pores and having antiseptic properties, to release agent from the zeolite and increase the content of agent in at least a part of the fluid and wherein the fluid contains molecules being larger than the micro-pores of the zeolite and having less affinity, as defined, to the zeolite than the agent.

50. The method of claim 49, characterized in that the fluid is a therapeutically active compound or composition in a therapeutically effective amount and/or concentration.

51. The method of claim 49, characterized in any characteristic of the preceding claims.

52. An arrangement for controlling microbial content or growth in a fluid, comprising a) an upstream chamber or conduit, b) a downstream chamber or conduit, c) a bed arranged between the upstream chamber and the downstream chamber in a manner allowing passage of the fluid at least from the upstream chamber through the bed to the downstream chamber, the bed comprising a zeolite with micro-pores charged with an agent having affinity to the zeolite micro-pores and having antiseptic properties and d) the fluid being present in at least one of the upstream chamber, the bed and the downstream chamber, characterized in the improvement comprising that the amount of agent charged to the zeolite is adapted to give release of agent to an antiseptic concentration of the agent in the fluid when in contact with the bed.

53. The arrangement of claim 52, characterized in any characteristic of the preceding claims.

54. An arrangement for controlling microbial content or growth in a fluid, comprising a) an upstream chamber or conduit, b) a downstream chamber or conduit, c) a bed arranged between the upstream chamber and the downstream chamber in a manner allowing passage of the fluid at least from the upstream chamber through the bed to the downstream chamber, the bed comprising a zeolite with micro-pores charged with an agent having affinity to the zeolite micro-pores and having antiseptic properties and d) the fluid being present in at least one of the upstream chamber, the bed and the downstream chamber, characterized in the improvement comprising that at least a volume of fluid downstream the bed contains an antiseptic concentration of the agent.

55. The arrangement of claim 54, characterized in any characteristic of the preceding claims.

56. A method for controlling microbial content or growth in a fluid, characterized in the steps of a) providing a bed of a zeolite with micro-pores, charged with an agent having affinity to the zeolite micro-pores and having antiseptic properties, and with macro-pores allowing passage of the fluid through the bed and b) passing at least a part of the fluid through the bed in a manner allowing release agent from the zeolite to increase the content of agent in at least a part of the fluid to an antiseptic concentration of the agent.

57. The method of claim 56, characterized in any characteristic of the preceding claims.

58. A hydrophobic zeolite with micro-pores, the zeolite having the general structure formula (AlO2)x(SiO2)y wherein the ratio y/x is at least 15, characterized in the improvement that the zeolite micro-pores are charged with an antiseptic agent in an amount corresponding to more than 10% of the zeolite maximum saturation amount for the agent and an absolute amount of more than 1% w/w to the zeolite.

59. The zeolite of claim 58, characterized in that the zeolite is charged with an antiseptic agent in an amount corresponding to more than 25%, preferably more than 35%, of the zeolite maximum saturation amount for the agent.

60. The zeolite of claim 58, characterized in that the zeolite is charged with an absolute amount of more than 5% w/w to the zeolite.

61. The zeolite of claim 58, characterized in the improvement that the charged zeolite is dry.

62. The zeolite of claim 58, characterized in any characteristic of the preceding claims.

63. Use of a hydrophobic zeolite with micro-pores, the zeolite having the general structure formula (AlO2)x(SiO2)y wherein the ratio y/x is at least 15, wherein the zeolite micro-pores are charged with an antiseptic agent, characterized in that, for the purpose of controlling microbial content or growth in a fluid, the fluid and charged zeolite are brought into contact to raise the concentration of antiseptic agent in the fluid to an antiseptically effective concentration

64. The use of claim 63, characterized in any characteristic of the preceding claims.

65. A method of controlling microbial content or growth in a fluid, characterized in the steps of a) providing a hydrophobic zeolite with micro-pores, the zeolite having the general structure formula (AlO2)x(SiO2)y wherein the ratio y/x is at least 15, wherein the zeolite micro-pores are charged with an antiseptic agent, and b) contacting the fluid with the zeolite to raise the concentration of antiseptic agent in at least a part of the fluid to an antiseptically effective concentration.

66. The method of claim 65, characterized in the step of maintaining the fluid in contact with the zeolite a time sufficient for antisptic action relative microbes.

67. The method of claim 65, characterized in that the fluid is removed and a new part of the fluid is contacted with the zeolite.

68. The method of claim 65, characterized in any characteristic of the preceding claims.

69. A method for controlling microbial content or growth in a fluid, characterized in the steps of a) contacting a first part of the fluid with a zeolite with micro-pores charged with an agent having affinity to the zeolite micro-pores and having antiseptic properties to release agent from the zeolite and increase the content of agent in at least a part of the first part of the fluid, b) maintaining the first part of the fluid in static contact with the zeolite under antiseptic conditions, c) removing the first part of the fluid from contact with the zeolite, having a reduced content of agent, and d) contacting a second part of the fluid with the zeolite, having said reduced content of agent.

70. The method of claim 69, characterized in any characteristic of the preceding claims.

71. A method for controlling microbial content or growth in a fluid, characterized in the steps of a) contacting a first part of the fluid with a zeolite with micro-pores charged with an agent having affinity to the zeolite micro-pores and having antiseptic properties to release agent from the zeolite and increase the content of agent in at least part of the fluid, b) removing the first part of the fluid from contact with the zeolite, c) separating at least a part of the agent from the first part of the fluid, e) adding said at least a part of agent to a second part of the fluid and d) contacting said second part of the fluid with the zeolite.

72. The method of claim 71, characterized in any characteristic of the preceding claims.
